(19) United States
(12) Patent Application Publication
(10) Pub. No.: US 2015/0078963 A1
(43) Pub. Date: Mar. 19, 2015

(54) Analysis Device

(71) Applicant: CERAGEM MEDISYS INC., Chungcheongnam-do (KR)

(72) Inventors: Jin Woo Lee, Chungcheongnam-do (KR); Jae Kyu Choi, Chungcheongnam-do (KR)

(21) Appl. No.: 14/385,811

(22) PCT Filed: Mar. 20, 2013
(86) PCT No.: PCT/KR13/02312

§ 371 Date: Sep. 17, 2014

(30) Foreign Application Priority Data
Mar. 21, 2012 (KR) 10-2012-0029011

(51) Int. Cl. G01N 33/53

(52) U.S. Cl. CPC G01N 33/5302
USPC 422/82.01

(57) ABSTRACT

Disclosed is an analysis device wherein a storage container for storing sensors is mounted in a main body, and automatic withdrawal, automatic measurement and automatic expulsion of the sensors are possible. The present invention comprises: a main body on the inside of which is mounted a storage container for housing a plurality of sensors in radial form; a drive means which is formed on the inside of the main body and causes the storage container to rotate such that the sensors are sequentially positioned in a discharge position; and a moving means which moves a sensor positioned in the discharge position, and moves same to a measurement position formed in the main body. In this way, there is automatic sensor supply and analysis.

### TECHNICAL FIELD

The present invention relates to an analysis device, and more particularly, to an analysis device in which a storage container storing a plurality of sensors therein is replaceably installed, and the sensors stored in the storage container are discharged, in turn, to perform an analyzing operation.

### BACKGROUND ART

Analysis devices for chemical and biochemical analysis of solid and liquid sample materials, and storages containers used in the analysis devices have been developed in research laboratories. Although a reaction associated with a sensitive reagent is complicated, a sensor based on a specially developed dry chemistry is not complicated, and thus may be used by a layman. As a main example of the sensor, there is a sensor for measuring a blood-sugar level of a diabetic patient. There are also sensors for performing various kinds of analysis operations, and analysis devices using the sensors. In the present invention, as an example, the sensor for measuring the blood will be described.

In general, a dry chemical sensor storing container is packed several times for a sale. The sensor is packed with a first package which directly covers the sensor, and an additional package (an external package). The first package is to satisfy an essential function of maintaining functions of chemical and biochemical components of a test element. The essential function is to protect the test element from an influence of light, or an introduction of air moisture, filth, fine substances and dirt, and also from a mechanical damage.

Generally, a conventional sensor packing sheet is sold in a state wrapped by the piece. When a user performs a blood analysis operation, after one of the sensors wrapped by piece is unwrapped and then inserted into an analysis device, blood is injected thereinto, and then the analysis operation is performed.

Since the conventional sensor is wrapped by the piece, there is a difficulty in storage, and it is inconvenient for the user to manually insert the sensors one by one into the analysis device. Further, while the user inserts the sensor by hand, the sensor may be contaminated, or may not be inserted into a proper position. Therefore, there are some problems in that an inaccurate result value may be obtained, and also a process thereof is inconvenient.

### DISCLOSURE

### Technical Problem

The present invention is directed to providing an analysis device in which a storage container storing a plurality of sensors therein is replaceably installed, and while the sensors are automatically withdrawn in turn, an analyzing operation is performed.

Also, the present invention is directed to providing an analysis device in which the sensors are withdrawn, in turn, from the storage container, and then automatically moved to a measurement position to perform the analyzing operation.

### Technical Solution

One aspect of the present invention provides an analysis device including a main body in which a cylindrical storage container having a plurality of sensors provided therein is installed; a driving means formed in the main body to rotate the storage container and thus to locate the sensors, in turn, at a discharging position; and a moving means configured to move the sensors located at the discharging position to a measurement position formed at the main body, wherein the moving means pushes the sensors through a cut-away portion formed at an outer circumferential surface of the storage container.

The storage container may be formed in a cylindrical shape in which the sensors are radially arranged, the cut-away portion which is in communication with a sensor storing part storing the sensors may be formed at the outer circumferential surface thereof, an opening may be formed at an upper end surface of one side thereof, and the cut-away portion and the opening may be sealed by a packing material.

The driving means may include a rotational shaft inserted into a coupling hole formed at a center of the storage container, a driving motor connected to the rotational shaft to rotate the rotational shaft, and a control member which controls the rotation of the rotational shaft to locate the sensors stored in the storage container, in turn, at the discharging position.

The control member may include an electrode plate attached to a rotational plate formed at one side of the rotational shaft and having an electrode groove formed along a circumferential surface, and an electrode terminal which is temporarily in contact with the electrode plate.

The moving means may include a driving motor installed in the main body, a pressing piece moved by the driving motor and configured to move the sensors stored in the storage container to a measurement position, and a guide member configured to guide the pressing piece.

The pressing piece may have a blade part formed at a distal end of one side thereof to be inserted into the storage container to move the sensors, a rotational pin formed at a distal end of the other side thereof and rotatably installed at the moving plate moved by the driving motor, and a guide protrusion provided at a middle portion thereof.

The moving plate may be slidably installed at the guide member, and an elastic member which presses the pressing piece toward the storage container may be installed at the moving plate.

A guide groove configured to guide the guide protrusion may be provided at the guide member, and the guide groove may include a moving section in which the blade part is inserted into the storage container to move the sensor to the measurement position, a discharging section which extends from a distal end of the moving section and in which the blade part discharges the sensor located at the measurement position to the outside of the main body, and a returning section which extends from the discharging section and guides the guide protrusion to an initial position of the moving section while the blade part is not in contact with the storage container.

A guide plate which guides the guide protrusion entering the discharging section toward the returning section may be installed at a distal end of the moving section.

The guide plate may include a plate part arranged along a lower surface of the returning section, and a bent part bent at a distal end of the plate part to have elasticity and in contact with a distal end of a lower surface of the moving section.

A cover configured to open and close the storage container installed in the main body may be installed at the main body, a discharging port in which the measurement position of the sensor is provided may be formed at the cover, and a recognition electrode may be provided at the measurement position.

A display part configured to display information and an input part configured to input a control command or information may be provided at the outer circumferential surface of the main body.

### Advantageous Effects

In the analysis device according to the present invention, since the storage container in which the plurality of sensors are stored is installed therein, and the sensors are automatically withdrawn to perform the sample analyzing operation, user's convenience is enhanced.

Also, in the analysis device according to the present invention, since the sensor is withdrawn in the state in which the storage container is installed in the main body, and the analyzing operation is performed, the sensors are prevented from being damaged while being moved, and also prevented from being contaminated due to the breakage of the packing material, and thus the accuracy of the analyzing operation can be enhanced.

### DESCRIPTION OF DRAWINGS

FIG. 1 is a perspective view illustrating an analysis device according to one embodiment of the present invention.

FIG. 2 is an exploded perspective view illustrating a storage container installing part.

FIG. 3 is a detail view illustrating an electrode plate of FIG. 2.

FIG. 4 is an exploded view illustrating the storage container installing part of the analysis device.

FIG. 5 is a state view illustrating an operating process of a pressing piece.

FIG. 6 is a view illustrating a state before the pressing piece moves a sensor.

FIG. 7 is a view illustrating a state in which the pressing piece moves the sensor to a measurement position.

FIG. 8 is a view illustrating a state in which the pressing piece discharges the sensor from the measurement position.

### MODES OF THE INVENTION

Hereinafter, an analysis device according to preferred embodiments of the present invention will be described in detail with reference to the accompanying drawings.

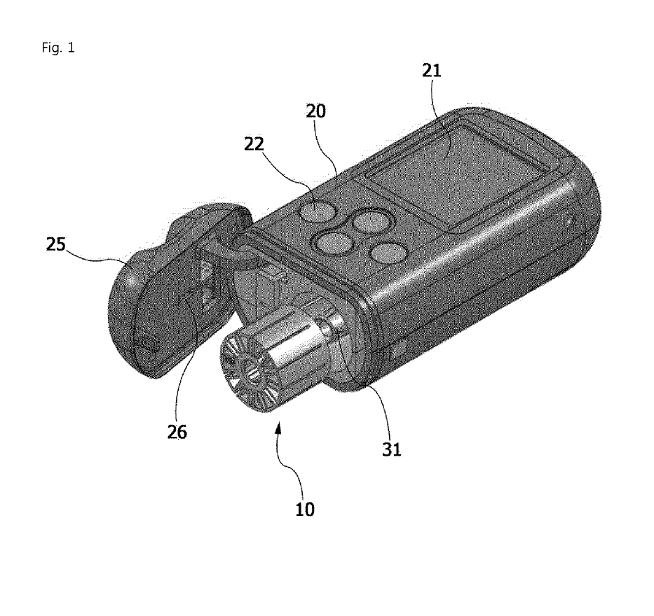

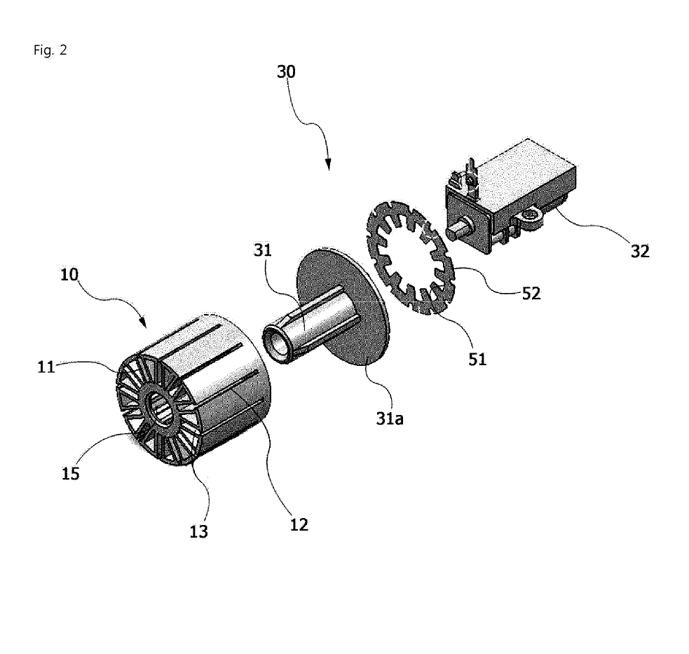

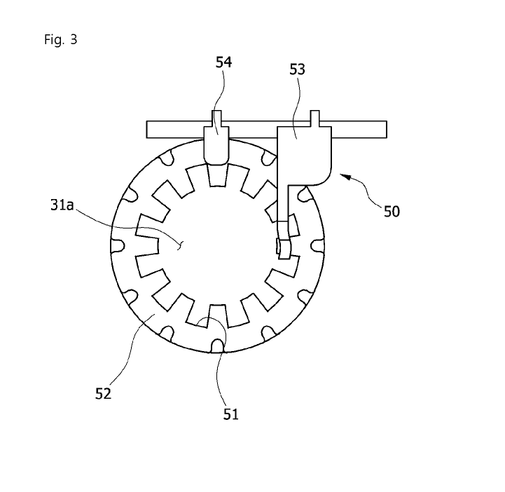

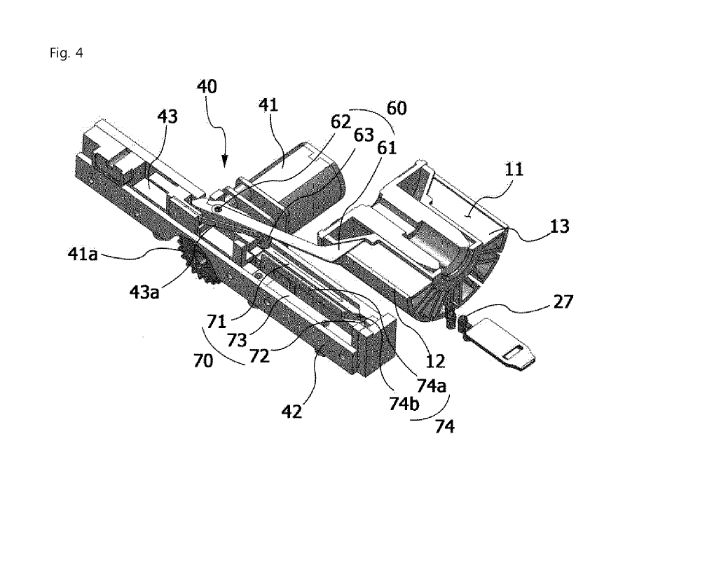

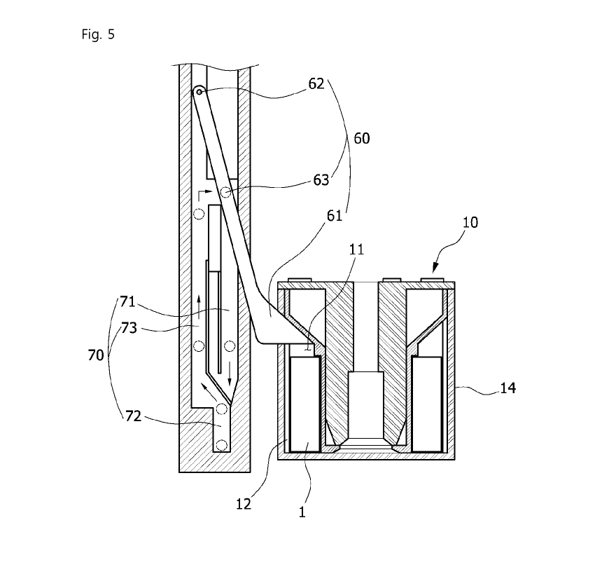

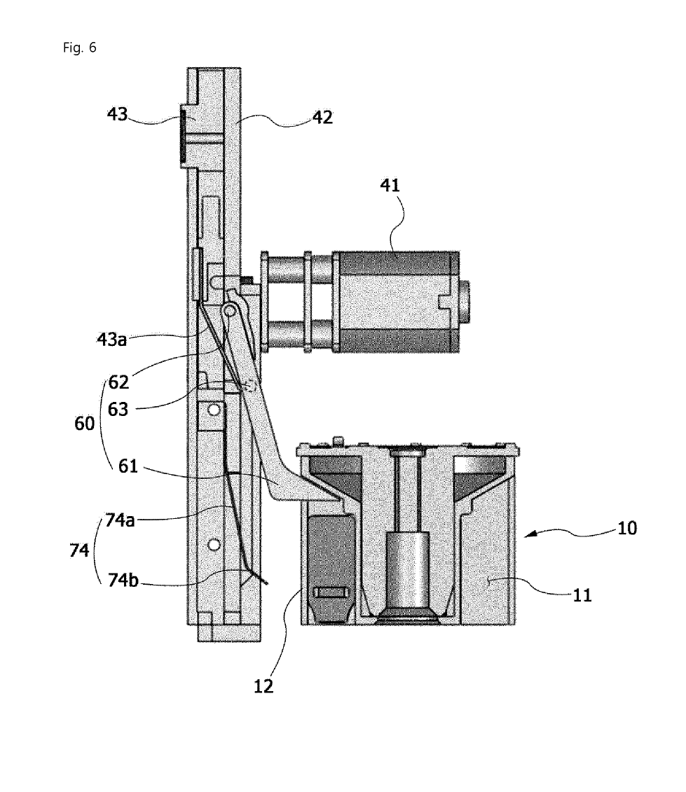

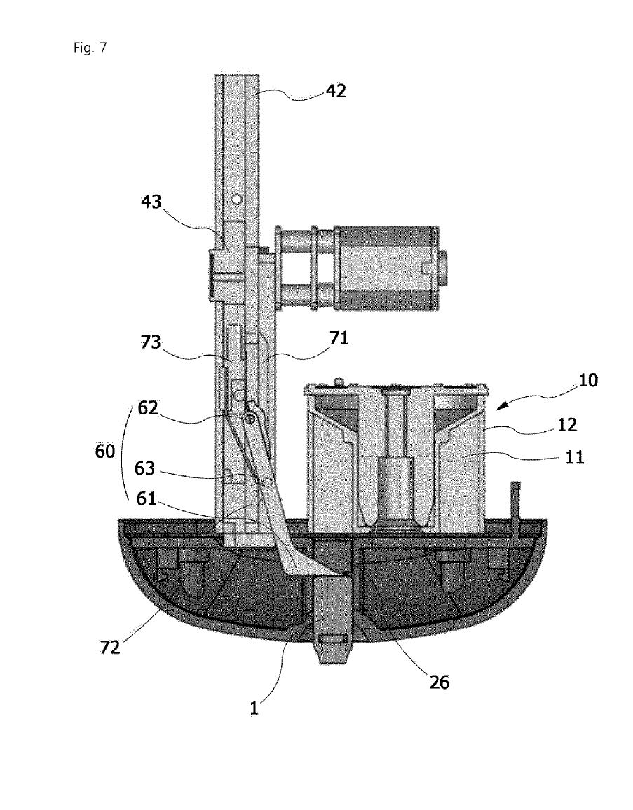

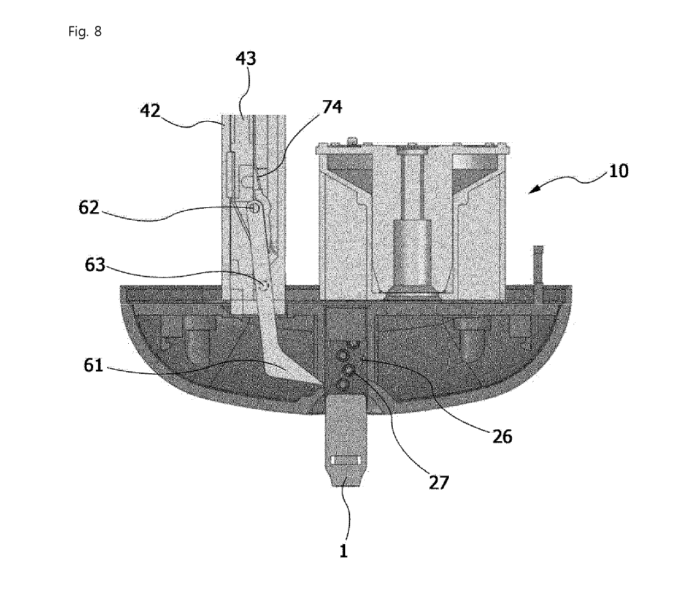

FIG. 1 is a perspective view illustrating an analysis device according to one embodiment of the present invention, FIG. 2 is an exploded perspective view illustrating a storage container installing part, FIG. 3 is a detail view illustrating an electrode plate of FIG. 2, FIG. 4 is an exploded view illustrating the storage container installing part of the analysis device, FIG. 5 is a state view illustrating an operating process of a pressing piece, FIG. 6 is a view illustrating a state before the pressing piece moves a sensor, FIG. 7 is a view illustrating a state in which the pressing piece moves the sensor to a measurement position, and FIG. 8 is a view illustrating a state in which the pressing piece discharges the sensor from the measurement position.

As illustrated in FIGS. 1 to 8, the analysis device according to the present invention has a structure in which a storage container 10 is installed in an internal receiving space of a main body 20 having a cover 25 provided at one side thereof.

The storage container 10 is formed in a cylindrical shape, and a packing material 14 is covered on an outer circumferential surface thereof. The storage container 10 has a plurality of sensor storing parts 11 such that a sensor 1 may be inserted thereinto. The sensor storing parts 11 are radially arranged with respect to a center point of the storage container 10. An opening 13 for communicating the sensor storing parts 11 with the outside is formed at an upper end surface of one side of the storage container 10. A cut-away portion 12 which extends and is cut away from a distal end of the other side to the opening 13 is provided at the outer circumferential surface of the storage container 20 to be in communication with the sensor storing parts 11.

The cut-away portion 12 has a predetermined gap in which a pressing piece 60 to be described later may be inserted. Preferably, the cut-away portion 12 extends horizontally with a central axis in a lengthwise direction.

The sensor storing parts 11 have a predetermined internal space in which the sensor 1 may be inserted. Preferably, a bottom surface provided at a distal end of the other side of the sensor storing parts 11 is formed to have an inclined surface. Preferably, the packing material 14 is formed of a thin film material, such as foil, which obstructs light and moisture and does not have an influence on characteristics of the sensor 1. Further, the inside of the storage container 10 is sealed with the packing material 14 in a state in which a moisture-proofing material is provided therein. A coupling hole 15 is formed to pass through the central axis of the storage container 10. The coupling hole 15 is a place in which a rotational shaft 31 formed at a measuring device is inserted. Preferably, a plurality of gear protrusions are formed at an inner circumferential surface of the coupling hole 15. The storage container 10 may further include an identification means which may confirm a kind of the stored sensor 1 or characteristics of a correction code or the like. The identification means may be installed at a lower end surface which is the other side of the storage container 10, but is not limited thereto. The identification means may be at least one of at least one electrode, a bar code, a QR code, a three-dimensional shape (or an arrangement or a combination of the shape), a code key, a color notifying means, an active/passive RF chip, a memory, and an identification element equivalent thereto.

The cover 25 is rotatably coupled to one side of the main body 20. The storage container 10 is installed in the internal receiving space opened and closed by the cover 25. A discharging port 26 having a measurement position of the sensor 1 is formed at the cover 25, and a recognition electrode 27 is provided at the measurement position. The recognition electrode 27 may be configured with at least two spring pins, may recognize an insertion of the sensor 1, whether a proper amount of sample is injected, and the code key, and also may electrically connect the sensor 1 for measuring the sample with a measuring circuit. In the drawings, the cover 25 is provided at a lower end portion of the main body 20 to be open and closed, but not limited thereto. For example, the cover 25 may be formed at an upper end portion of the main body 1. Alternatively, the cover 25 may be formed at a side surface or a lower surface of the main body 1. In this case, the cover serves as only a door for an installation space of the storage container 10, and thus an installation structure of the storage container 10 or a discharging structure of the sensor 1 may be properly modified.

A display part 21 displaying information and a button type input part 22 inputting a control command or information are provided at the outer circumferential surface of the main body 20. A structure in which the display part 21 and the input part 22 are separately provided is described as an example, but another structure, like a touch pad used in an electronic product such as a smart phone or copy machine, in which inputting and displaying of the information are integrated into one, may be provided.

The sensor 1 is an analyzing unit or expendables for analysis such as an electrochemical strip, an optical strip and an immunodiagnostic strip, and is preferably used as a disposable part. The sensor 1 may include a sample introduction part and a reaction part, and may further include a reaction transferring part which transfers the reaction, or a reaction displaying part which displays the reaction. As an example, the electrochemical strip includes a capillary tube for introduction of the sample, an electrode part in which a reaction reagent is fixed, and a signal transferring part which transfers a reaction signal.

A driving means 30 which rotates the storage container 10 and locates the sensor 1 or the sensor storing parts 11, in turn, at a discharging position, and a moving means 40 which moves the sensor 1 located at the discharging position to the measurement position formed at the cover 25 of the main body 20 are formed in the body 20. Further, although not illustrated, a controlling means or circuit part which controls an operation, a displaying operation, a sample measuring operation, a result calculating operation, driving of a program, or the like, of construction units including the driving means 30 and the moving means 40 is formed in the main body 20. It should be noted that driving of all units is controlled by the controlling means or the circuit part.

The driving means 30 includes the rotational shaft 31 inserted into the coupling hole 15 formed at a center of the storage container 10, a driving motor 32 connected to the rotational shaft 31 to rotate the rotational shaft 31, and a control member 50 which controls a rotation of the rotational shaft 31. A rotational plate 31a is attached to a distal end of one side of the rotational shaft 31.

The control member 50 includes an electrode plate 52 formed in a circular ring shape attached to the rotational plate 31a, and electrode terminals 53 and 54 which are in contact with the electrode plate 52. The electrode plate 52 is formed in the circular ring shape, such that electrode grooves 51 are formed to be recessed along an inner circumferential surface at regular intervals. The electrode terminal 53 is installed to be, in turn, in contact with the electrode grooves 51 of the rotated electrode plate 52, and another electrode terminal 54 is installed to be in continuous contact with the rotated electrode plate 52. The rotation of the storage container 10 may be controlled in a manner in which the motor 32 is stopped at a moment when the electrode terminal 53 is located at the electrode groove 51 and a current is disconnected, and thus the sensor 1 is located at the discharging position, or in a manner in which the motor 32 is stopped at a moment when the electrode terminal 53 escapes from the electrode groove 51 and the current is connected, and thus the sensor 1 is located at the discharging position.

The moving means 40 includes a driving motor 41 installed in the main body 20 and having a pinion gear 41a, a moving plate 43 having a rack gear formed at one side surface thereof to be engaged with the pinion gear 41a and slidably installed at a guide member 42, the pressing piece 60 rotatably installed at the moving plate 43, and the guide member 42 configured to guide the moving plate 43 and the pressing piece 60. The moving plate 43 may be moved in various manners other than a structure using the pinion gear 41a and the rack gear, for example, by transmitting power of the driving motor 41 through a link, a wire, or the like.

The pressing piece 60 may be formed in a plate shape having a predetermined width and length, and is preferably formed of a metallic material. The pressing piece 60 has a blade part 61 formed at a distal end of one side thereof, a rotational pin 62 formed at a distal end of the other side thereof, and a guide protrusion 63 formed at a middle portion thereof. Preferably, a distal end of the blade part 61 is formed to be sharp and thus to tear the packing material 14 and enter therein, and the blade part 61 is formed to be bent and thus to be in close contact with a front surface of one side of the sensor 1. The rotational pin 62 is formed in a cylindrical shape which protrudes in a thickness-wise direction, and rotatably installed at the moving plate 43. The guide protrusion 63 protrudes in a thickness-wise direction, and is slid while being in contact with a guide groove 70 formed at the guide member 42. The guide protrusion 63 is formed of a separate material such as a plastic material, and may be attached to the pressing piece 60. Further, an elastic member 43a which presses the rotatably coupled pressing piece 60 toward the storage container 10 is installed at the moving plate 43.

In the guide groove 70 formed at the guide member 42 to guide the guide protrusion 63, a moving section 71 in which the blade part 61 is inserted into the storage container 10 to move the sensor 1 from an initial position to a measurement position, a discharging section 72 which extends from a distal end of the moving section 71 and in which the blade part 61 discharges the sensor 1 located at the measurement position to the outside of the main body 20, and a returning section 73 which extends from the discharging section 72 and guides the guide protrusion 63 to the initial position of the moving section 71 while the blade part 61 is not in contact with the storage container 10, are formed in a closed-section shape. The moving section 71 and the returning section 73 are arranged to be layered, and the moving section 71 is located adjacent to the storage container 10. A guide plate 74 which guides the guide protrusion 63 entering the discharging section 72 toward the returning section 73 is installed at the distal end of the moving section 71. The guide plate 74 includes a plate part 74a arranged along a lower surface of the returning section 73, and a bent part 74b bent at a distal end of the plate part 74a to have elasticity and in contact with a distal end of a lower surface of the moving section 71.

An operating process of the analysis device according to one embodiment of the present invention is as follows.

If the cover 25 is opened and the storage container 10 is installed, a position of the first sensor storing part 11 of the storage container 10 is aligned with a position of the discharging port 26 by an operation of the driving means 30. At this time, the aligned position is recognized by an operation of the control member 50.

In this state, when an operation is started by pressing a button of the input part 22, the moving means 40 is driven, and as illustrated in FIGS. 5 and 6, the moving plate 43 is moved along the guide member 42 in a lengthwise direction, and at the same time, the guide protrusion 63 is moved along the moving section 71. If the guide protrusion 63 is moved along the moving section 71, the pressing piece 60 is rotated around the rotational pin 62, and the blade part 61 tears the packing material 14 and enters the inside of the sensor storing part 11 of the storage container 10.

Then, as illustrated in FIG. 7, in a state in which the blade part 61 enters the sensor storing part 11, the moving plate 43 is continuously moved, and, when the guide protrusion 63 is moved to the distal end of the moving section 71, the blade part 61 pushes the sensor 1 to be discharged through the opening 13 of the storage container 10, and then the sensor 1 is introduced into the discharging port 26 formed at the cover 25 and located at the measurement position. At the measurement position, one part of the sensor 1 is in contact with a socket and electrically connected with the measuring unit, and the other part thereof is exposed to suck a sample.

At the measurement position, if measuring and (qualitative or quantitative) analyzing processes of the sensor are completed, the operation of the moving means 40 is resumed, and as illustrated in FIG. 8, the moving plate 43 is further moved, and thus the guide protrusion 63 of the pressing piece 60 enters the discharging section 72. The guide protrusion 63 entering the discharging section 72 presses the bent part 74b of the guide plate 74 at the distal end of the moving section 71, and the guide plate 74 is elastically deformed, and the guide protrusion 63 deviates from the guide plate 74 and enters the discharging section 72.

The guide protrusion 63 entering the discharging section 72 is moved to the distal end of the discharging section 72, and thus the blade part 61 of the pressing piece 60 pushes the sensor 1 to be discharged to the outside of the cover 25.

If the discharging of the sensor 1 is completed, the driving motor 41 is reversely rotated. The moving plate 43 is moved in a reverse direction by the rotation of the driving motor 41, and the guide protrusion 63 of the pressing piece 60 is moved along the discharging section 72 in the reverse direction. While moving along the discharging section 72, the guide protrusion 63 is moved along the bent part 74b of the guide plate 74 to the returning section 73.

If the guide protrusion 63 is moved along the returning section 73, the pressing piece 60 is rotated, and the blade part 61 is returned in a state of being not in contact with the storage container 10.

In a state in which the guide protrusion 63 is moved to the distal end of the returning section 73, the operation is finished. If an operating signal is input through the input part 22, the next position of the sensor storing part 11 of the storage container 10 is aligned with the position of the discharging port 26 by the operation of the driving means 30, and the guide protrusion 63 is moved to an initial position of the moving section 71, and the next analyzing operation is started.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. An analysis device comprising:
a main body (20) in which a cylindrical storage container (10) having a plurality of sensors (1) provided therein is installed;
a driving means (30) formed in the main body (20) to rotate the storage container (10) and thus to locate the sensors (1), in turn, at a discharging position; and
a moving means (40) configured to move the sensors (1) located at the discharging position to a measurement position formed at the main body (20),
wherein the moving means (40) pushes the sensors (1) through a cut-away portion (12) formed at an outer circumferential surface of the storage container (10).

2. The analysis device of claim 1, wherein the storage container (10) is formed in a cylindrical shape in which the sensors (10) are radially arranged, the cut-away portion (12) which is in communication with a sensor storing part (11) storing the sensors (1) is formed at the outer circumferential surface thereof, an opening (13) is formed at an upper end surface of one side thereof, and the cut-away portion (12) and the opening (13) are sealed by a packing material (14).

3. The analysis device of claim 1, wherein the driving means (30) comprises a rotational shaft (31) inserted into a coupling hole (15) formed at a center of the storage container (10), a driving motor (32) connected to the rotational shaft (31) to rotate the rotational shaft (31), and a control member (50) which controls a rotation of the rotational shaft (31) to locate the sensors (1) stored in the storage container (10), in turn, at the discharging position.

4. The analysis device of claim 3, wherein the control member (50) comprises an electrode plate (52) attached to a rotational plate (31a) formed at one side of the rotational shaft (31) and having an electrode groove (51) formed along a circumferential surface, and an electrode terminal (53) which is temporarily in contact with the electrode plate (52).

5. The analysis device of claim 1, wherein the moving means (40) comprises a driving motor (41) installed in the main body (20), a pressing piece (60) moved by the driving motor (41) and configured to move the sensors (1) stored in the storage container (10) to a measurement position, and a guide member (42) configured to guide the pressing piece (60).

6. The analysis device of claim 5, wherein the pressing piece (60) has a blade part (61) formed at a distal end of one side thereof to be inserted into the storage container to move the sensors, a rotational pin (62) formed at a distal end of the other side thereof and rotatably installed at the moving plate (43) moved by the driving motor (41), and a guide protrusion (63) provided at a middle portion thereof.

7. The analysis device of claim 6, wherein the moving plate (43) is slidably installed at the guide member (42), and an elastic member (43a) which presses the pressing piece (60) toward the storage container (10) is installed at the moving plate (43).

8. The analysis device of claim 6, wherein a guide groove (70) configured to guide the guide protrusion (63) is provided at the guide member (42), and the guide groove (70) comprises a moving section (71) in which the blade part (61) is inserted into the storage container (10) to move the sensor (1) to the measurement position, a discharging section (72) which extends from a distal end of the moving section (71) and in which the blade part (61) discharges the sensor (1) located at the measurement position to the outside of the main body (20), and a returning section (73) which extends from the discharging section (72) and guides the guide protrusion (63) to an initial position of the moving section (71) while the blade part (61) is not in contact with the storage container (10).

9. The analysis device of claim 8, wherein a guide plate (74) which guides the guide protrusion (63) entering the discharging section (72) toward the returning section (73) is installed at a distal end of the moving section (71).

10. The analysis device of claim 9, wherein the guide plate (74) comprises a plate part (74a) arranged along a lower surface of the returning section (73), and a bent part (74b) bent at a distal end of the plate part (74a) to have elasticity and in contact with a distal end of a lower surface of the moving section (71).

11. The analysis device of claim 1, wherein a cover (25) configured to open and close the storage container (20) installed in the main body (20) is installed at the main body (20), a discharging port (26) in which the measurement position of the sensor (1) is provided is formed at the cover (25), and a recognition electrode (27) is provided at the measurement position.

12. The analysis device of claim 1, wherein a display part (21) configured to display information and an input part (22) configured to input a control command or information are provided at an outer circumferential surface of the main body (20).
